(19) United States
(12) Patent Application Publication
(10) Pub. No.: US 2004/0122327 A1
(43) Pub. Date: Jun. 24, 2004

(54) Intrauterine imaging system

(76) Inventors: Amir Belson, Cupertino, CA (US); Doron Kreiser, Herzlia (IL)

Correspondence Address: LEARY & ASSOCIATES, 3900 NEWPARK MALL RD., THIRD FLOOR, SUITE 317, NEWARK, CA, 94560, US

(21) Appl. No.: 10/152,164

(22) Filed: May 20, 2002

Related U.S. Application Data
(63) Continuation-in-part of application No. 10/024,656, filed on Dec. 17, 2001.
(60) Provisional application No. 60/256,155, filed on Dec. 15, 2000.

(51) Int. Cl. A61B 5/05; A61B 6/00

(52) U.S. Cl. 600/476

(57) ABSTRACT

An obstetrical imaging system includes a uteroscope or video-uteroscope for intrauterine imaging, a control unit for operating the uteroscope, a computer imaging station with a video monitor for viewing the images captured by the optical imaging system and a recording system for recording the images. The uteroscope includes an optical imaging system, which preferably includes a panoramic lens for viewing the entire uterine cavity in one image, mounted on an elongated shaft for insertion into the patient's uterus. One or more transparent inflatable balloons are mounted on the elongated shaft surrounding the optical imaging system. An instrument channel is provided in the shaft of the uteroscope for insertion of instruments, such as a suction tube, external to or in between the transparent inflatable balloons.

### CROSS REFERENCE TO OTHER APPLICATIONS

This application is a continuation-in-part of U.S. patent application Ser. No. 10/024,656, filed on Dec. 17, 2001, which claims the benefit of U.S. Provisional Patent Application, serial No. 60/256,155, filed on Dec. 15, 2000.

### FIELD OF THE INVENTION

The present invention relates generally to medical imaging systems and, more particularly, to an obstetrical imaging system that includes a uteroscope or video-uteroscope for intrauterine imaging.

### BACKGROUND OF THE INVENTION

Adequate diagnosis and intervention during labor are crucial to reduce maternal and fetal morbidity and mortality that are inherent risks of vaginal deliveries. Modern obstetrics is still considered to be an “art” instead of a “science”, as it is based on the operators' subjective impression and experience rather than analysis of objective clinical findings. The evaluation of the labor process is based on interpretation of digital pelvic examination. Moreover, residents who are still acquiring their skills perform most of the public deliveries in the U.S. Regardless, even the most experienced operators have up to thirty percent error rate.

The risks to the mother during labor and delivery do not end with the expulsion of the baby. Postpartum complications are caused by rupture of the uterus and more commonly by retained parts of the placenta in the uterine cavity. Similar problems involving retained products of conception in the uterine cavity can be encountered in elective termination of pregnancy or abortion. Retained products of conception (RCP) is a common complication that can be found in histological specimen in 10-15% of normal non-caesarian births and 3-5% of abortions. RPC not extracted may cause intense bleeding from the uterus, intrauterine infections resulting in intrauterine adhesions, long term effects such as infertility and exposure of the medical staff to negligence litigations.

Current attempts to detect RPC include ultrasonic imaging postpartum partum uterine cavity manual revision and hysteroscopy. Ultrasonic imaging suffers from low sensitivity and specificity due to artifacts caused by air and blood. It requires a high level of operator skill. It lacks the ability to image and document the entire uterine cavity in one picture. Post partum uterine cavity manual revision is performed by insertion of the physician's hand into the uterine cavity and a manual exploration for the uterine wall defect or the retained parts of the placenta. Uterine cavity manual revision is highly painful for the patient and demands profound analgesia or sedation. The procedure requires a high level of operator skill and, even when performed by skilled operators, is not highly reliable. Furthermore, it lacks the ability to image and document the result. Hysteroscopy requires a highly skilled operator for success, in particular due to the narrow field of view that it provides. The procedure depends on expending the uterine cavity with fluid or gas that leaks through the open cervix. It cannot be done acutely post partum and it lacks the ability to document the entire uterine cavity in one picture.

It would be desirable, therefore, to provide apparatus and methods for diagnostic examination and treatment of retained products of conception and other conditions in a patient's uterine cavity. The apparatus and methods should be safe, simple, reliable and painless and should be adaptable to the normal uterus and also to the post partum uterus. Preferably, the apparatus and methods will provide an ability to image and document the entire uterine cavity in one picture.

### SUMMARY OF THE INVENTION

In keeping with the foregoing discussion, the present invention provides an obstetrical imaging system that includes a uteroscope or video-uteroscope for intrauterine imaging. The uteroscope includes an optical imaging system mounted on an elongated shaft configured for insertion into the patient's uterus. One or more transparent inflatable balloons are mounted on the elongated shaft surrounding the optical imaging system. Preferably, the uteroscope includes a panoramic lens that allows simultaneous one-shot imaging of the entire uterine cavity. The elongated shaft of the uteroscope may be rigid, semi-rigid or flexible and may be articulated or steerable. An instrument channel is provided in the shaft of the uteroscope for insertion of instruments, such as a suction tube, external to or in between the transparent inflatable balloons.

The obstetrical imaging system may also include a control unit for operating the uteroscope, a computer imaging station with a video monitor for viewing the images captured by the optical imaging system and a recording system for recording the images. Computer hardware and/or software may be provided as part of the system for simultaneous one-shot imaging of the entire uterine cavity and for viewing the images obtained. Alternatively or in addition, the obstetrical imaging system may include a video monitor connected to or integrated with the uteroscope.

### BRIEF DESCRIPTION OF THE DRAWINGS

FIG. 1 shows an obstetrical imaging system according to the present invention, including a video-uteroscope, a control unit and a computer imaging station.

FIG. 2 shows the video-uteroscope being inserted in a deflated state into a patient's uterus.

FIG. 3 shows the video-uteroscope with the transparent balloon inflated within the patient's uterine cavity.

FIG. 4 represents the panoramic imaging capability of the video-uteroscope for imaging and documenting the patient's entire uterine cavity in one picture.

FIG. 5 shows a rigid video-uteroscope with a single transparent inflatable balloon inflated within a post partum uterus.

FIG. 6 shows an articulable flexible video-uteroscope with a single transparent inflatable balloon inflated within a post partum uterus.

FIG. 7 shows a diagnostic video-uteroscope with multiple transparent inflatable balloons inflated within a post partum uterus.

FIG. 8 shows a therapeutic video-uteroscope system with multiple transparent inflatable balloons inflated within a post partum uterus.

### DETAILED DESCRIPTION OF THE INVENTION

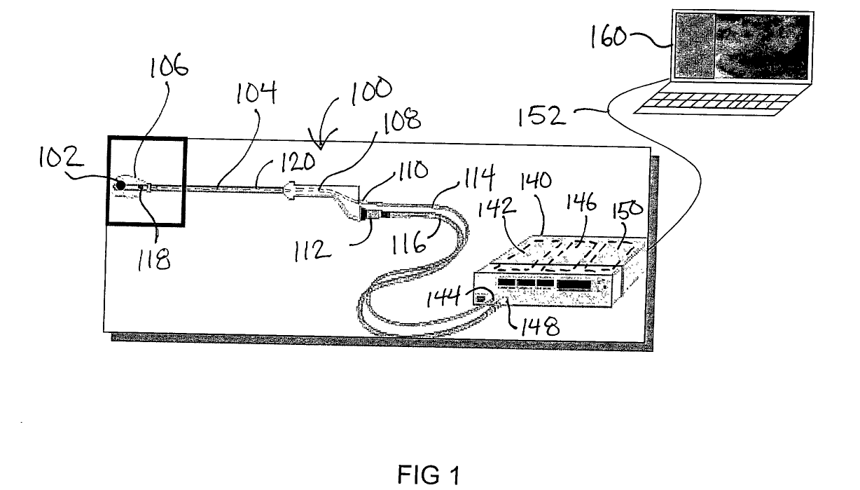

FIG. 1 shows an obstetrical imaging system constructed according to the present invention. The obstetrical imaging system includes a uteroscope or video-uteroscope 100, a control unit 140 and a computer imaging station 104. The uteroscope 100 includes an optical imaging system 102 mounted on an elongated shaft 104 configured for insertion into the patient's uterus. One or more transparent inflatable balloons 106 are mounted on the elongated shaft 104 surrounding the optical imaging system 102. The elongated shaft 104 of the uteroscope 100 may be rigid, semi-rigid or flexible and may be articulated or steerable. For therapeutic applications of the uteroscope 100, an instrument channel is provided in the shaft 104 for insertion of instruments, such as a suction tube, external to or in between the transparent inflatable balloons 106. The uteroscope 100 will preferably include a proximal handle 108 with an inflation/deflation fitting 110 and a video connector 112 for connecting the uteroscope 100 to the control unit 140 via an inflation/deflation tube 114 and a video cable 116. An inflation lumen 120 extends through the shaft 104 of the uteroscope 100 from the inflation/deflation fitting 110 to an inflation port 118 in fluid communication with the interior of the balloons 106.

The control unit 140 includes an inflation/deflation controller 142 with a fitting 144 for connecting to the uteroscope 100 via the inflation/deflation tube 114. In one exemplary embodiment, the inflation/deflation controller 142 includes a pressure regulator and an electrically controlled valve circuit connected to a source of inflation fluid, such as compressed carbon dioxide gas or another physiologically acceptable fluid, for inflating the balloons 106 and to a vacuum source for deflating the balloons 106. In another exemplary embodiment, the inflation/deflation controller 142 would include a motor-driven syringe pump or the like for inflating and deflating the balloons 106.

Preferably, the control unit 140 also includes a video controller 146 and an illumination source 150 with a fitting 148 for connecting to the uteroscope 100 via the video cable 116. The video controller 146 includes circuitry for operating the optical imaging system 102 and for initial processing of the video images captured. The video controller 146 is connected to the computer imaging station 160 via a second video cable 152. Alternatively or in addition, the obstetrical imaging system may include a video monitor connected to or integrated with the uteroscope. Preferably, the system includes a recording device, which may be located within the control unit 140 or the computer imaging station 104, for recording the images captured by the uteroscope 100.

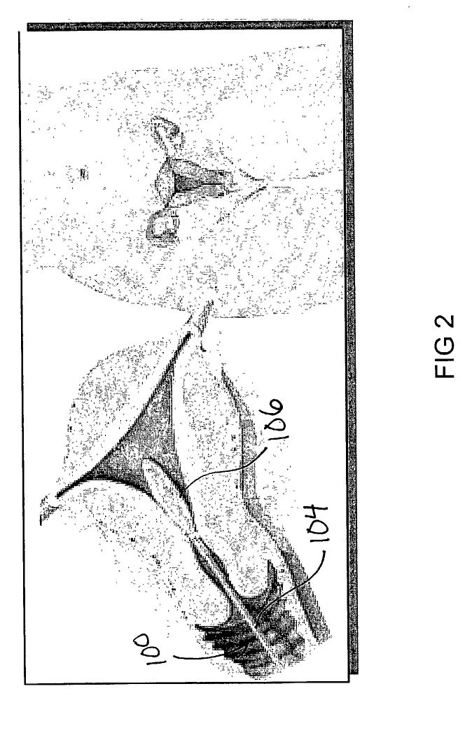

FIG. 2 shows the video-uteroscope 100 being inserted through the vagina and cervix into the patient's uterus with the balloons 106 in a deflated state. When in the deflated state, the inflatable balloon or balloons 106 are collapsed against and/or wrapped around the elongated shaft 104 to minimize the insertion profile of the device. Preferably, the inflation/deflation controller 142 draws a vacuum through the inflation lumen 120 to maintain the balloons 106 in the deflated state during insertion.

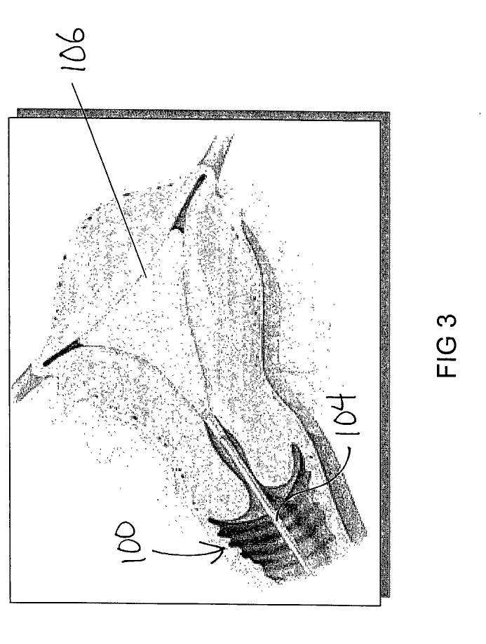

FIG. 3 shows the video-uteroscope 100 with the transparent balloon or balloons 106 inflated within the patient's uterine cavity. Preferably, the transparent balloon or balloons 106 are of thin-walled polymeric construction and inflatable at low pressure to substantially fill the uterine cavity without dilating or distending it. The material of the balloon or balloons 106 may be an elastomer with a low modulus of elasticity that allows it to conform to the interior of the uterine cavity when inflated without dilating or distending it. Alternatively, the balloon or balloons 106 may be configured as a flaccid bag with a volume greater than that of the uterine cavity so that, when inflated, it substantially fills the uterine cavity without dilating or distending it.

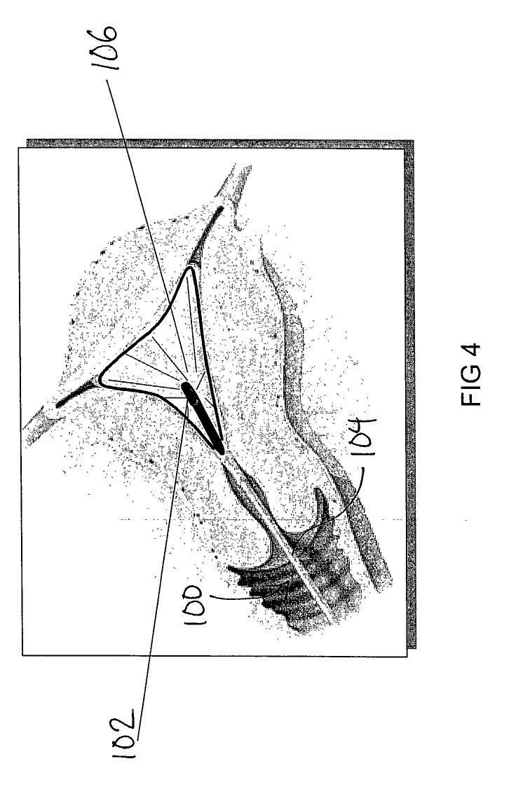

Preferably, the optical imaging system 102 of the video-uteroscope 100 is capable of panoramic imaging to capture the image of the entire uterine cavity in one picture. FIG. 4 graphically illustrates the panoramic imaging capability of the video-uteroscope 100 for imaging and documenting the patient's entire uterine cavity in one picture. For this purpose, the uteroscope employs a spherical lens or equivalent optical element that provides a panoramic image so that a large portion of the uterine wall can be viewed at once with a minimum of manipulation of the uteroscope. In a particularly preferred embodiment, the spherical lens provides a panoramic image of the entire uterine wall at once. Alternatively or in addition, the uteroscope 100 and/or the optical imaging system 102 may be configured to manually or automatically scan the interior of the uterine cavity using either a spherical lens or ordinary lens to capture images of the entire uterine cavity that can be combined into a panoramic image by the computer imaging station 104. The panoramic image can be viewed in real time and/or the image data can be captured electronically for later viewing, archiving or image manipulation. The image of the uterine wall can be viewed on the display monitor of the computer imaging station 104 in a panoramic mode or the image can be electronically panned and zoomed to examine various portions of the uterine wall in detail. In addition, the image can be electronically manipulated to eliminate any distortion caused by the spherical lens. The imaging system can also be used to create a map projection of the image so that the entire uterine wall can be viewed with a minimum of image distortion.

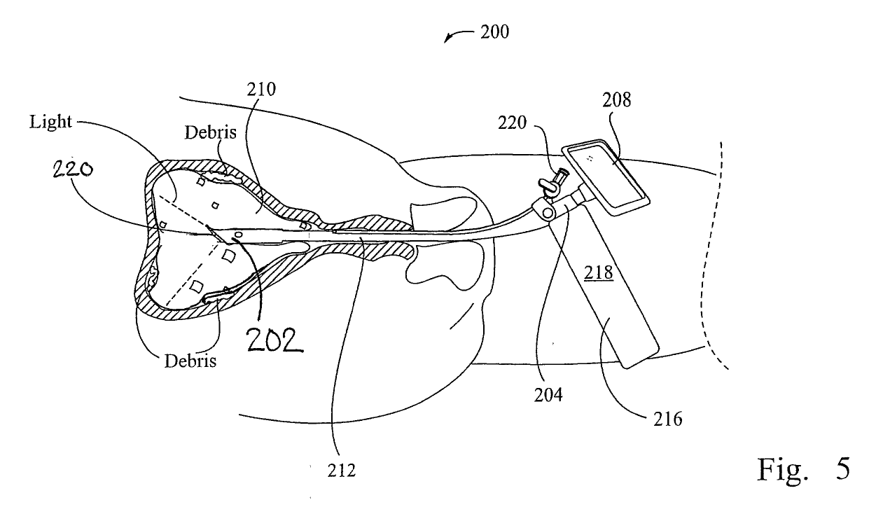

FIG. 5 shows a video-uteroscope with a single transparent inflatable balloon inflated within a post partum uterus. The uteroscope 200 has a rigid or semi-rigid shaft 212 with a miniaturized video camera 202 mounted at its distal end, a fiber optic light source 204 and a display monitor 208. The transparent inflatable balloon 210 surrounds a distal portion of the uteroscope 200. In this exemplary embodiment, the light source 204, the display monitor 208 and a replaceable or rechargeable battery 216 have been integrated into the handle 218 of the uteroscope 200. The uteroscope 200 is inserted into the uterus after delivery and the transparent balloon 210 is inflated via an inflation lumen 220 by a pressure controlled pump, preferably using carbon dioxide gas or a physiologic solution, to create a clear field for imaging the uterine walls. Preferably, the miniaturized video camera 202 of the uteroscope 200 includes a spherical lens or other optical element for panoramic imaging to capture the image of the entire uterine cavity in one picture. Alternatively, the miniaturized video camera 202 may have an ordinary lens, which is preferably mounted at an angle with respect to the shaft 212 of the uteroscope 200 so that the entire interior of the uterus can be viewed by rotating the uteroscope 200. The uteroscope 200 can be used to identify damage to the uterine wall or retained portions of the placenta and to diagnose other problems. In an alternative embodiment, the rigid uteroscope 200 may include fiber optics or rod optics to transmit images to a video camera connected to the proximal end of the shaft 212.

The lens 220 of the miniaturized video camera 202 may be located on the interior of the transparent inflatable balloon 210. Alternatively, to avoid direct contact between the miniaturized video camera 202 and the inflation fluid, the lens 220 of the miniaturized video camera 202 may be position within an invagination in the wall of the inflatable balloon 210 or inside of a balloon-within-a-balloon so that the lens 220 is isolated from the interior of the transparent inflatable balloon 210.

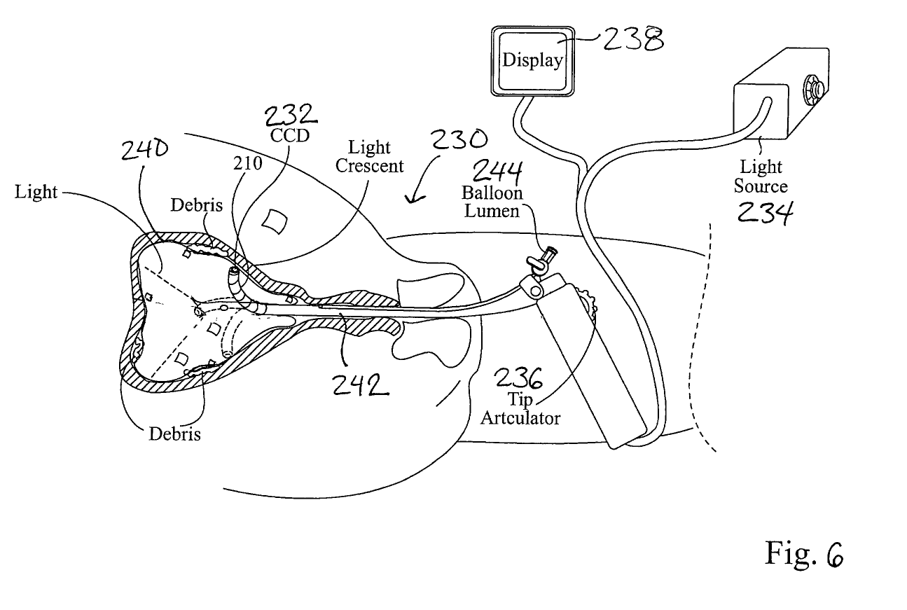

FIG. 6 shows an articulable flexible video-uteroscope 230 with a single transparent inflatable balloon 240 inflated within a post partum uterus. The uteroscope 230 has a flexible articulable shaft 242 with a miniaturized video camera 232 mounted at its distal end, a fiber optic light source 234 and a display monitor 238. The transparent inflatable balloon 240 surrounds a distal portion of the uteroscope 230. In this exemplary embodiment, the light source 234 and the display monitor 238 are separate from the uteroscope 230. The uteroscope 230 is inserted into the uterus after delivery and the transparent balloon 240 is inflated via an inflation lumen 244 by a pressure controlled pump, preferably using carbon dioxide gas or a physiologic solution, to create a clear field for imaging the uterine walls. The flexible shaft 242 of the video uteroscope 230 can be rotated and/or articulated with the tip articulator knob 236 to view the entire interior of the uterus. The uteroscope 230 can be used to identify damage to the uterine wall or retained portions of the placenta and to diagnose other problems.

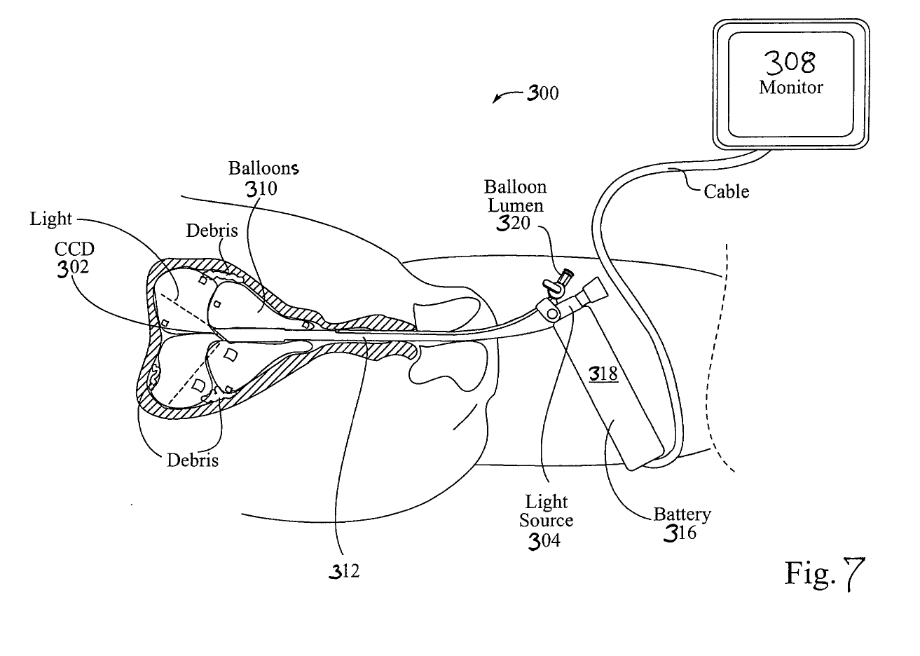

FIG. 7 shows a diagnostic video-uteroscope 300 with multiple transparent inflatable balloons 310 inflated within a post partum uterus. The uteroscope 300 has a rigid or semi-rigid shaft 312 with a miniaturized video camera 302 mounted at its distal end, a fiber optic light source 304 and a display monitor 308. The transparent inflatable balloons 310 surround a distal portion of the uteroscope 300. In this exemplary embodiment, the light source 304 and a replaceable or rechargeable battery 316 have been integrated into the handle 318 of the uteroscope 300 and the display monitor 308 is separate from the uteroscope 300. The uteroscope 300 is inserted into the uterus after delivery and the transparent balloons 310 are inflated via an inflation lumen 320 by a pressure controlled pump, preferably using carbon dioxide gas or a physiologic solution, to create a clear field for imaging the uterine walls. Alternatively, the uteroscope 300 may be configured with a multiplicity of inflation lumens for separately and independently inflating each of the transparent balloons 310.

Preferably, the transparent balloons 310 are all of thin-walled polymeric construction and inflatable at low pressure to fill the uterine cavity without dilating or distending it. The material of the balloons 310 may be an elastomer with a low modulus of elasticity that allows them to conform to the interior of the uterine cavity when inflated without dilating or distending it. Alternatively, the balloons 310 may be configured as flaccid bags with a total volume greater than that of the uterine cavity so that, when inflated, they fill the uterine cavity without dilating or distending it. Preferably, the uteroscope 300 includes a spherical lens or other optical element for panoramic imaging to capture the image of the entire uterine cavity in one picture. Alternatively, the miniaturized video camera 302 may have an ordinary lens, which is preferably mounted at an angle with respect to the shaft 312 of the uteroscope 300 so that the entire interior of the uterus can be viewed by rotating the uteroscope 300. The uteroscope 300 can be used to identify damage to the uterine wall or retained portions of the placenta and to diagnose other problems. In an alternative embodiment, the rigid uteroscope 300 may include fiber optics or rod optics to transmit images to a video camera connected to the proximal end of the shaft.

Preferably, the miniature video camera 302 is located between the transparent inflatable balloons 310 such that, when inflated, the balloons 310 surround the video camera 302. Alternatively, the miniature video camera 302 may be located on the interior of one of the transparent inflatable balloons 310 or inside of a balloon-within-a-balloon so that the video camera 302 is isolated from the inflation fluid within the interior of the transparent inflatable balloon 310.

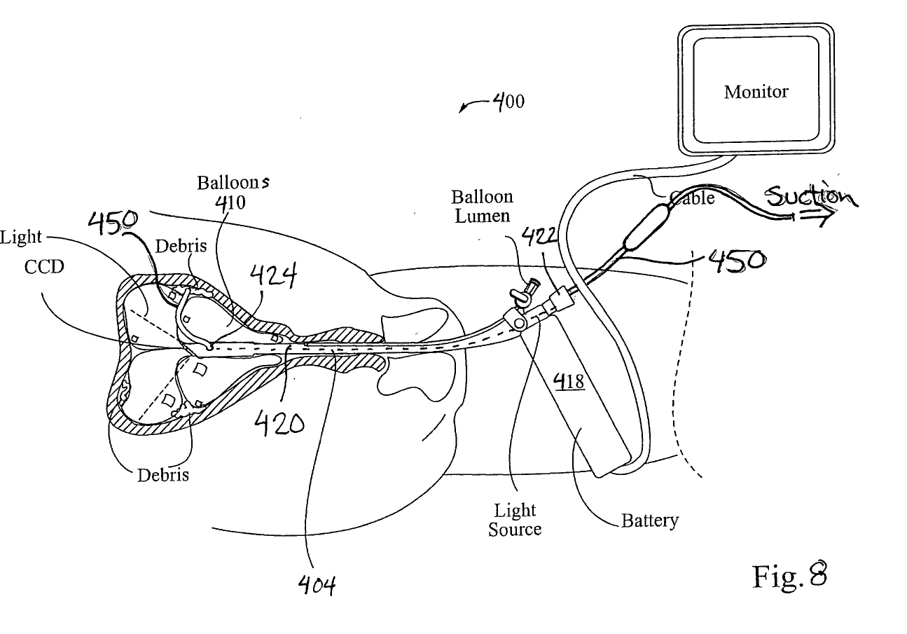

FIG. 8 shows a therapeutic video-uteroscope 400 with multiple transparent inflatable balloons 410 inflated within a post partum uterus. The therapeutic video-uteroscope 400 is similar in construction to the diagnostic video-uteroscope 300 of FIG. 7, with the addition of an instrument channel 420 in the shaft 404 for insertion of instruments, such as a suction tube 450 for aspirating any retained material within the uterus. The instrument channel 420 extends from a proximal instrument port 422 on the handle 418 of the uteroscope 400 to a distal instrument port 424 near the distal end of the shaft 404 in between the transparent inflatable balloons 410. The suction tube 450 can be manipulated independently of the uteroscope 400 and can move between and/or displace the inflated balloons 410 to access any part of the patient's uterus.

Each of the various embodiments of the obstetrical imaging system may be configured as a piece of durable equipment to be resterilized and reused. Alternatively, some or all of the obstetrical imaging system may be configured as a disposable product for one-time use only.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof. For example, many of the features described can be used together in combinations other than those explicitly described.

What is claimed is:

1. An intrauterine imaging system, comprising:
an optical imaging system mounted on an elongated shaft configured for insertion into a uterine cavity of a patient; and
a transparent inflatable balloon mounted on the elongated shaft surrounding the optical imaging system, wherein the transparent inflatable balloon is configured to substantially fill the uterine cavity when inflated.

2. The intrauterine imaging system of claim 1, wherein the optical imaging system comprises a miniaturized video camera.

3. The intrauterine imaging system of claim 1, wherein the elongated shaft is rigid.

4. The intrauterine imaging system of claim 1, wherein the elongated shaft is flexible.

5. The intrauterine imaging system of claim 1, wherein the elongated shaft is articulable.

6. The intrauterine imaging system of claim 1, further comprising a display monitor for displaying images of the interior of the patient's uterine cavity from the optical imaging system.

7. The intrauterine imaging system of claim 1, wherein the optical imaging system is equipped with a panoramic lens for simultaneously imaging a large portion of the interior of the patient's uterine cavity.

8. The intrauterine imaging system of claim 7, further comprising a computer imaging station with a display monitor for displaying a panoramic image of the interior of the patient's uterine cavity.

9. The intrauterine imaging system of claim 7, wherein the computer imaging station is configured with pan and zoom functions for displaying images of selected portions of the interior of the patient's uterine cavity on the display monitor.

10. An intrauterine imaging system, comprising:
an optical imaging system mounted on an elongated shaft configured for insertion into a uterine cavity of a patient; and
a multiplicity of transparent inflatable balloons mounted on the elongated shaft surrounding the optical imaging system.

11. The intrauterine imaging system of claim 10, wherein the multiplicity of inflatable balloons are configured to substantially fill the uterine cavity when inflated.

12. The intrauterine imaging system of claim 10, wherein the optical imaging system comprises a miniaturized video camera.

13. The intrauterine imaging system of claim 10, further comprising a display monitor for displaying images of the interior of the patient's uterine cavity from the optical imaging system.

14. The intrauterine imaging system of claim 10, wherein the optical imaging system is equipped with a panoramic lens for imaging a large portion of the interior of the patient's uterine cavity.

15. The intrauterine imaging system of claim 14, further comprising a computer imaging station with a display monitor for displaying a panoramic image of the interior of the patient's uterine cavity.

16. The intrauterine imaging system of claim 15, wherein the computer imaging station is configured with pan and zoom functions for displaying images of selected portions of the interior of the patient's uterine cavity on the display monitor.

17. The intrauterine imaging system of claim 10, further comprising an instrument channel within the elongated shaft terminating in a distal instrument port positioned between the multiplicity of inflatable balloons.

18. The intrauterine imaging system of claim 17, further comprising a suction tube sized and configured for insertion through the instrument channel.
